(19) United States
(12) Patent Application Publication
(10) Pub. No.: US 2021/0030816 A1
(43) Pub. Date: Feb. 4, 2021

(54) FEED INGREDIENT COMPRISING CLOSTRIDIUM TYROBUTYRICUM

(71) Applicant: WHITE DOG LABS, INC., New Castle, DE (US)

(72) Inventors: Bryan P. TRACY, Wilmington, DE (US); Shawn William JONES, Bear, DE (US); Biniam MARU, New Castle, DE (US); Aharon M. EYAL, Jerusalem (IL)

(21) Appl. No.: 17/067,784

(22) Filed: Oct. 12, 2020

Related U.S. Application Data
(63) Continuation-in-part of application No. PCT/US2019/026996, filed on Apr. 11, 2019.
(60) Provisional application No. 62/657,309, filed on Apr. 13, 2018.
(60) Provisional application No. 62/731,988, filed on Sep. 17, 2018.

(51) Int. Cl. A61K 35/742; A23K 10/18; A61K 47/12; A61K 35/747; A61K 35/744

(52) U.S. Cl. CPC A61K 35/742; A23K 10/18; A61K 35/744; A61K 35/747; A61K 47/12

(57) ABSTRACT

Provided is a feed ingredient comprising at least 20% by weight Clostridium tyrobutyricum, wherein said Clostridium tyrobutyricum comprises at least 70% of dry weight crude protein and optionally at least 7% of dry weight lysine and/or at least 1% of dry weight methionine. Further provided are animal feeds and probiotic compositions comprising the feed ingredient, methods for the production thereof and uses thereof.

### CROSS-REFERENCE TO RELATED APPLICATIONS

This patent application is a continuation-in-part of PCT international application number PCT/US2019/026996, having an international filing date of Apr. 11, 2019, published as international publication number WO 2019/200103 A1, which is hereby incorporated by reference in its entirety; which claims benefit and priority from U.S. 62/657,309, filed on Apr. 13, 2018 and U.S. 62/731,988 filed on Sep. 17, 2018, both of which are hereby incorporated by reference in their entirety.

### BACKGROUND OF THE INVENTION

There is an on-going need for new feed ingredients of improved feed qualities, including higher crude protein content and higher content of essential amino acids.

### SUMMARY OF THE INVENTION

According to an embodiment, provided is a feed ingredient comprising at least 20% by weight Clostridium tyrobutyricum, wherein said Clostridium tyrobutyricum comprises at least 70% of dry weight crude protein and optionally at least 7% of dry weight lysine and/or at least 1% of dry weight methionine. According to an embodiment, said Clostridium tyrobutyricum is genetically modified. According to an embodiment, said Clostridium tyrobutyricum is adaptively evolved.

According to an embodiment, said feed ingredient further comprises at least 20% by weight Clostridium pasteurianum. According to an embodiment, said feed ingredient further comprises at least 20% by weight an acetogenic bacteria. According to an embodiment, said feed ingredient further comprises at least 20% by weight Butyribacterium methylotrophicum. According to an embodiment, said feed ingredient further comprises at least 10% by weight Clostridium pasteurianum and at least 10% by weight Butyribacterium methylotrophicum.

According to an embodiment, said feed ingredient is characterized by water solubility greater than 25% by weight. According to an embodiment, said Clostridium tyrobutyricum is sporulated. According to an embodiment, said Clostridium tyrobutyricum is in clostridial form.

According to an embodiment, provided is an animal feed comprising said feed ingredient. According to an embodiment, said feed ingredient further comprises at least one of butyric acid and propionic acid. According to an embodiment, provided is an aqua feed comprising said feed ingredient.

According to an embodiment, provided is a probiotic preparation comprising said feed ingredient. According to an embodiment, provided is a poultry probiotic preparation comprising said feed ingredient. According to an embodiment, provided is a swine probiotic preparation comprising said feed ingredient. According to an embodiment, provided is a companion animal probiotic preparation comprising said feed ingredient. According to an embodiment, provided is a human probiotic preparation comprising said feed ingredient. According to an embodiment, said probiotic preparation further comprising at least one of Enterococcus faecium, Bacillus subtilis, Bacillus amyloliquefaciens, Lactobacillus acidophilus, Lactobacillus plantarum, Pediococcus acidilactici and Clostridium butyricum.

According to an embodiment, provided is a method for producing said feed ingredient, comprising culturing in a fermentation medium Clostridium tyrobutyricum, whereby Clostridium tyrobutyricum cell mass is formed. According to an embodiment, said fermentation medium is kept at a pH greater than 5.25.

According to an embodiment, said method further comprises co-culturing Clostridium pasteurianum, whereby said cell mass further comprises Clostridium pasteurianum. According to an embodiment, said method further comprises co-culturing an acetogenic bacteria, whereby said cell mass further comprises an acetogenic bacteria. According to an embodiment, said method further comprises co-culturing Butyribacterium methylotrophicum, whereby said cell mass further comprises Butyribacterium methylotrophicum. According to an embodiment, said method further comprises co-culturing Clostridium pasteurianum and Butyribacterium methylotrophicum, whereby said cell mass further comprises Clostridium pasteurianum and Butyribacterium methylotrophicum. According to an embodiment, said method further comprises mechanical separation of formed cell mass and at least one of washing, drying, pulverizing and pelletizing.

According to an embodiment, provided is a method for maintaining the good health of an animal comprising feeding said animal an animal feed comprising said feed ingredient. According to an embodiment, said animal is selected from the group consisting of finfish and shellfish, larvae, fry, juvenile, and adult fish, poultry, swine, companion animals, other ruminants, and humans. According to an embodiment, said maintaining good health comprises at least one of reduced susceptibility to enteric pathogens, reduction in bowel inflammation, reduced mortality, improved feed conversion and improved weight gain.

According to an embodiment, provided is a method for maintaining the good health of an animal comprising feeding said animal a probiotic preparation comprising said feed ingredient. According to an embodiment, said animal is selected from the group consisting of finfish and shellfish, larvae, fry, juvenile, and adult fish, poultry, swine, companion animals, other ruminants and humans. According to an embodiment, said maintaining good health comprises at least one of reduced susceptibility to enteric pathogens, reduction in bowel inflammation, reduced mortality, improved feed conversion and improved weight gain.

### DETAILED DESCRIPTION OF THE INVENTION

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

As used herein, the term “feed ingredient” refers to a component, part, constituent or any combination/mixture in an animal feed.

As used herein, the term “animal feed” refers to a product intended for consumption by an animal, such as livestock, poultry, companion animals and the like. The term “animal feed” as used herein is intended to include food intended for human consumption.

As used herein, the term “probiotic” refers to a live microorganism which provides health benefits to an animal when consumed, generally by restoring the balance of gut flora.

As used herein, the term “maintaining good health” refers to regulating a system of the body, such as by treating, preventing or reducing incidence and/or severity and/or duration of known conditions as compared to the incidence and/or severity of such conditions in the absence of use of the method as disclosed herein.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Additional advantages of the invention will be set forth in part in the description, which follows, and in part will be obvious from the description, or may be learned by practice of the invention. It is to be understood that both the foregoing general description and the following detailed description are exemplary and explanatory only and are not restrictive of the invention, as claimed.

According to an embodiment, provided is a feed ingredient comprising at least 20% by weight (20% wt) Clostridium tyrobutyricum, wherein said Clostridium tyrobutyricum comprises at least 70% of dry weight crude protein and optionally at least 7% of dry weight lysine and/or at least 1% of dry weight methionine. According to an embodiment, provided is a feed ingredient comprises at least 30% wt, at least 40% wt, at least 50% wt, at least 60% wt, at least 70% wt, at least 80% wt, at least 90% wt or at least 95% wt Clostridium tyrobutyricum. According to an embodiment, said Clostridium tyrobutyricum comprises at least 75% of dry weight crude protein, at least 80%, at least 85% or at least 90%. According to an embodiment, said Clostridium tyrobutyricum comprises at least 7% of dry weight lysine, at least 7.5%, at least 8%, at least 8.5% or at least 9%. According to an embodiment, said Clostridium tyrobutyricum comprises at least 1% of dry weight methionine, at least 1.5%, at least 2%, or at least 2.5%.

According to an embodiment, said feed ingredient further comprises at least 20% wt Clostridium pasteurianum, at least 30% wt, at least 40% wt, at least 50% wt, at least 60% wt or at least 70% wt. According to an embodiment, said feed ingredient comprises at least 1% wt glycogen, at least 5% wt, at least 10% wt, at least 15% wt, at least 20% wt, at least 25% wt or at least 30% wt. According to an embodiment, said feed ingredient further comprises at least 20% wt Butyribacterium methylotrophicum, at least 30% wt, at least 40% wt or at least 50% wt.

According to an embodiment, said feed ingredient further comprises at least 20% wt of acetogenic bacteria, at least 30% wt, at least 40% wt, at least 50% wt, at least 60% wt or at least 70% wt.

According to an embodiment, said feed ingredient further comprises at least 20% wt Butyribacterium methylotrophicum at least 30% wt, at least 40% wt, at least 50% wt, at least 60% wt or at least 70% wt.

According to an embodiment, said feed ingredient further comprises at least 10% wt Clostridium pasteurianum, at least 20% wt or at least 30% wt and at least 10% by weight Butyribacterium methylotrophicum, at least 20% wt or at least 30% wt.

According to an embodiment, said animal feed is characterized by water solubility greater than 25% wt, greater than 40% wt, greater than 60% wt, greater than 80% wt, or greater than 80% wt.

According to an embodiment, said Clostridium tyrobutyricum is sporulated. According to an embodiment, said Clostridium tyrobutyricum is in clostridial form.

According to an embodiment, said Clostridium tyrobutyricum is genetically modified. According to an embodiment, said Clostridium tyrobutyricum is genetically modified by removing or inserting genetic material out of or into the chromosome. According to an embodiment, said genetic modification is not plasmid-based. According to an embodiment, said Clostridium tyrobutyricum is genetically modified to produce propionic acid. According to an embodiment, said Clostridium tyrobutyricum is genetically modified to reduce the production of granulose. According to an embodiment, said Clostridium tyrobutyricum is adaptively evolved. According to an embodiment, said Clostridium tyrobutyricum is adaptively evolved to have a higher growth rate. According to an embodiment, said Clostridium tyrobutyricum is adaptively evolved to have an improved substrate utilization rate. According to an embodiment, said Clostridium tyrobutyricum is adaptively evolved to have a higher crude protein content.

According to an embodiment, said clostridial form is characterized by the formation of granulose. According to an embodiment, said granulose comprises amylopectin and optionally also amylose.

According to an embodiment, provided is an animal feed comprising said feed ingredient. According to an embodiment, said feed ingredient forms at least 2% wt of said animal feed, at least 4% wt, at least 6% wt, at least 8% wt or at least 10% wt. According to an embodiment, said animal feed further comprises at least one of butyric acid and propionic acid. According to an embodiment, said animal feed further comprises amylopectin and amylose with amylopectin to amylose weight/weight ratio of about 1 to 4.

According to an embodiment, provided is an aqua feed comprising said feed ingredient. According to an embodiment, said feed ingredient forms at least 2% wt of said aqua feed, at least 4% wt, at least 6% wt, at least 8% wt or at least 10% wt.

According to an embodiment, provided is a probiotic preparation comprising said feed ingredient. According to an embodiment, said feed ingredient forms at least 20% wt of said probiotic preparation, at least 40% wt, at least 60% wt, at least 80% wt or at least 90% wt. According to an embodiment, said probiotic preparation further comprising at least one of Enterococcus faecium, Bacillus subtilis, Bacillus amyloliquefaciens, Lactobacillus acidophilus, Lactobacillus plantarum, Pediococcus acidilactici and Clostridium butyricum.

According to an embodiment, provided is a poultry probiotic preparation comprising said feed ingredient. According to an embodiment, said feed ingredient forms at least 20% wt of said poultry probiotic preparation, at least 40% wt, at least 60% wt, at least 80% wt or at least 90% wt.

According to an embodiment, provided is a human probiotic preparation comprising said feed ingredient. According to an embodiment, said feed ingredient forms at least 20% wt of said human probiotic preparation, at least 40% wt, at least 60% wt, at least 80% wt or at least 90% wt.

According to an embodiment, provided is a method for producing said feed ingredient, comprising culturing in a fermentation medium Clostridium tyrobutyricum, whereby fermentation broth comprising Clostridium tyrobutyricum cell mass is formed. According to an embodiment, said fermentation broth further comprises at least one of acetic acid and butyric acid. According to an embodiment, said fermentation medium is kept at a pH greater than 5.25, greater than 5.30, greater than 5.35, greater than 5.40, greater than 5.45, or greater than 5.50.

According to an embodiment, said method comprises co-culturing Clostridium pasteurianum, whereby said cell mass further comprises Clostridium pasteurianum. According to an embodiment, said method comprises co-culturing an acetogenic bacteria, whereby said cell mass further comprises an acetogenic bacteria. According to an embodiment, said method comprises co-culturing Butyribacterium methylotrophicum, whereby said cell mass further comprises Butyribacterium methylotrophicum. According to an embodiment, said method comprises co-culturing Clostridium pasteurianum and Butyribacterium methylotrophicum, whereby said cell mass further comprises Clostridium pasteurianum and Butyribacterium methylotrophicum. According to an embodiment, said fermentation medium comprises a first feedstock comprising carbohydrates, glycerol, methanol, or combinations thereof. According to an embodiment, said fermentation medium comprises a second feedstock comprising CO, CO₂, carbonate, bicarbonate, H₂, glycerol, methanol, formate, urea or mixtures thereof. According to an embodiment, said first feedstock, said second feedstock or both are metabolized during said culturing.

According to an embodiment, said method comprises mechanical separation of formed cell mass and at least one of washing, drying pulverizing, extrusion and pelletizing. According to an embodiment, said method comprises processing the cell mass to have a water solubility greater than 25%, greater than 50%, greater than 60%, greater than 70%, greater than 80%, or greater than 90%.

According to an embodiment, provided is a method for maintaining the good health of an animal comprising feeding said animal an animal feed comprising said feed ingredient. According to an embodiment, said animal is selected from the group consisting of finfish and shellfish, including larvae, fry, juvenile, and adult fish, poultry, swine, companion animals, such as dogs and cats, other ruminants and humans. According to an embodiment, said maintaining good health comprises at least one of reduced susceptibility to enteric pathogens, reduction in bowel inflammation, reduced mortality, improved feed conversion, and improved weight gain.

According to an embodiment, provided is a method for maintaining the good health of an animal comprising feeding said animal a probiotic preparation comprising said feed ingredient. According to an embodiment, said animal is selected from the group consisting of finfish and shellfish, including larvae, fry, juvenile, and adult fish, poultry, swine, companion animals, other ruminants and humans. According to an embodiment, said maintaining good health comprises at least one of reduced susceptibility to enteric pathogens, reduction in bowel inflammation, consumption of lactic acid, reduced mortality, improved feed conversion, and improved weight gain.

### EXAMPLES

### Example 1. Clostridium tyrobutyricum Fermentation

Clostridium tyrobutyricum was grown on a hydrolyzed corn starch medium with 10 mL/L trace elements solution (2.0 g/L nitrilotriacetic acid, 1.0 g/L MnSO₄.H₂O, 0.8 g/L Fe(SO₄)₂(NH₄)₂.6H₂O, 0.2 g/L CoCl₂.6H₂O, 0.2 mg/L ZnSO₄.7H₂O, 20.0 mg/L CuCl₂.2H₂O, 20.0 mg/L NiCL₂.6H₂O, 20.0 mg/L Na₂MoO₄.2H₂O, 20.0 mg/L Na₂SeO₄, and 20.0 mg/L Na₂WO₄) and 10 mL/L Wolfe's vitamins (2.0 mg/L biotin, 2.0 mg/L folic acid, 10.0 mg/L pyridoxine hydrochloride, 5.0 mg/L thiamine-HCl, 5.0 mg/L riboflavin, 5.0 mg/L nicotinic acid, 5.0 mg/L calcium D-(+)-pantothenate, 0.1 mg/L vitamin B12, 5.0 mg/L p-aminobenzoic acid, and 5.0 mg/L thioctic acid). The estimated glucose concentration was 65 g/L. The cells were grown under anaerobic conditions (low CO₂ sparge) at 37° C. The pH was bottom controlled at 5.5 with 8M NH₄OH. Table 1 shows the increase in optical density (OD) over time along with butyric acid.

TABLE 1
Clostridium tyrobutyricum fermentation.

| Fermentation | | Concentration (g/L) | | | |
|---|---|---|---|---|---|
| time (h) | OD* | Lactic acid | Acetic acid | Butyric acid | Ethanol |
| 0 | 0.44 | 0.54 | 0.18 | 0.21 | 0.09 |
| 6 | 2.53 | 0.29 | 0.48 | 1.08 | 0.16 |
| 12 | 11.50 | 0.88 | 1.46 | 4.72 | 0.29 |
| 18 | 36.30 | 0.10 | 2.47 | 10.09 | 0.41 |

*OD measured at 600 nm

At 18 hours, the cells were harvested, washed three times in water, and dried with a dual drum dryer. The resulting dried cell mass had the characteristics listed in Table 2.

TABLE 2
Characteristics of Clostridium tyrobutyricum cell mass.

| Characteristic | As is % | Dried % |
|---|---|---|
| Moisture | 4.7 | — |
| Crude protein | 79.9 | 83.9 |
| Ash | 7.85 | 8.24 |
| Lysine | — | 8.33 |
| Methionine | — | 1.42 |

### Example 2. Co-Culture Fermentation

A co-culture of Clostridium tyrobutyricum and Butyribacterium methylotrophicum was prepared and grown on hydrolyzed corn starch medium. The seed culture was generated by mixing equal volumes of a pure culture of C. tyrobutyricum and B. methylotrophicum. The estimated glucose concentration was 100 g/L. The cells were grown under anaerobic conditions (low CO₂ sparge) at 37° C. The pH was bottom controlled at 5.5 with 8M NH₄OH. Table 3 shows the increase in optical density (OD) over time along with butyric acid.

TABLE 3
Co-culture fermentation.

| Fermentation | | Concentration (g/L) | | | |
|---|---|---|---|---|---|
| time (h) | OD* | Lactic acid | Acetic acid | Butyric acid | Ethanol |
| 0 | 0.56 | 0.69 | 0.70 | 0.23 | 0.65 |
| 16 | 11.20 | 0.00 | 1.30 | 6.08 | 0.20 |
| 24 | 17.60 | 0.00 | 2.78 | 13.61 | 1.05 |
| 42 | 22.20 | 0.00 | 2.99 | 26.86 | 1.49 |

*OD measured at 600 nm

At 42 hours, the cells were harvested and washed three times in water. The resulting wet cell mass was analyzed and had the characteristics listed in Table 4.

TABLE 4
Characteristics of Clostridium tyrobutyricum cell mass.

| Characteristic | As is % | Dried % |
|---|---|---|
| Moisture | 69.1 | — |
| Crude protein | 24.5 | 79.3 |
| Ash | 1.8 | 5.8 |

### Examples 3-28

### Feed Ingredient Cell-Mass Compositions

| | Clostridium | Butyribacterium | Clostridium |
|---|---|---|---|
| | tyrobutyricum | methylotrophicum | pasteurianum |
| Example | (% wt) | (% wt) | (% wt) |
| 3 | 95 | <5 | <5 |
| 4 | 75 | 20 | <5 |
| 5 | 65 | 30 | <5 |
| 6 | 55 | 40 | <5 |
| 7 | 45 | 50 | <5 |
| 8 | 75 | <5 | 20 |
| 9 | 65 | <5 | 30 |
| 10 | 55 | <5 | 40 |
| 11 | 45 | <5 | 50 |
| 12 | 75 | 10 | 10 |
| 13 | 70 | 15 | 10 |
| 14 | 70 | 10 | 15 |
| 15 | 65 | 15 | 15 |
| 16 | 60 | 20 | 15 |
| 17 | 60 | 15 | 20 |
| 18 | 55 | 20 | 20 |
| 19 | 55 | 25 | 15 |
| 20 | 55 | 15 | 25 |
| 21 | 50 | 25 | 20 |
| 22 | 50 | 20 | 25 |
| 23 | 45 | 25 | 20 |
| 24 | 45 | 20 | 25 |
| 25 | 45 | 30 | 20 |
| 26 | 45 | 20 | 30 |
| 27 | 40 | 35 | 20 |
| 28 | 40 | 20 | 35 |

1. A feed ingredient comprising at least 20% by weight Clostridium tyrobutyricum, wherein said Clostridium tyrobutyricum comprises at least 70% of dry weight crude protein and optionally at least 7% of dry weight lysine and/or at least 1% of dry weight methionine.

2. The feed ingredient of claim 1, further comprising at least 20% by weights Butyribacterium methylotrophicum.

3. The feed ingredient of claim 1, wherein said Clostridium tyrobutyricum is sporulated.

4. The feed ingredient of claim 1, wherein said Clostridium tyrobutyricum is in clostridial form.

5. The feed ingredient of claim 1, wherein said Clostridium tyrobutyricum is genetically modified.

6. The feed ingredient of claim 1, wherein said Clostridium tyrobutyricum is genetically modified by removing or inserting genetic material out of or into the chromosome.

7. The feed ingredient of claim 1, wherein said Clostridium tyrobutyricum is adaptively evolved.

8. An animal feed comprising the feed ingredient according to claim 1.

9. The animal feed of claim 8, further comprising at least one of butyric acid and propionic acid.

10. A probiotic preparation comprising the feed ingredient according to claim 1.

11. The probiotic preparation of claim 10, being selected from the group consisting of a poultry probiotic preparation, a human probiotic preparation, a swine probiotic preparation, and a companion animal probiotic preparation.

12. The probiotic preparation of claim 10, further comprising at least one of Enterococcus faecium, Bacillus subtilis, Bacillus amyloliquefaciens, Lactobacillus acidophilus, Lactobacillus plantarum, Pediococcus acidilactici and Clostridium butyricum.

13. A method for producing a feed ingredient according to claim 1, comprising culturing in a fermentation medium Clostridium tyrobutyricum, whereby Clostridium tyrobutyricum cell mass is formed.

14. The method of claim 13, wherein said fermentation medium is kept at a pH greater than 5.25.

15. The method of claim 13, further comprising mechanical separation of formed cell mass and at least one of washing, drying, pulverizing and pelletizing.

16. A method for maintaining the good health of an animal comprising feeding said animal an animal feed according to claim 8.

17. The method of claim 16, wherein said animal is selected from the group consisting of finfish and shellfish, larvae, fry, juvenile, and adult fish, poultry, swine, companion animals, other ruminants and humans.

18. The method of claim 16, wherein said maintaining good health comprises at least one of reduced susceptibility to enteric pathogens, reduction in bowel inflammation, reduced mortality, improved feed conversion and improved weight gain.

19. A method for maintaining the good health of an animal comprising feeding said animal a probiotic preparation according to claim 10.

20. The method of claim 19, wherein said maintaining good health comprises at least one of reduced susceptibility to enteric pathogens, reduction in bowel inflammation, reduced mortality, improved feed conversion and improved weight gain.
